(19) United States
(12) Patent Application Publication
(10) Pub. No.: US 2025/0195807 A1
(43) Pub. Date: Jun. 19, 2025

(54) PATIENT INTERFACE DEVICE

(71) Applicant: DCSTAR INC., New York, NY (US)

(72) Inventor: David LUO, New York, NY (US)

(21) Appl. No.: 18/540,275

(22) Filed: Dec. 14, 2023

(51) Int. Cl. A61M 16/06

(52) U.S. Cl. CPC A61M 16/0616; A61M 16/0666; A61M 16/0683; A61M 2205/42

(57) ABSTRACT

A patient interface device to deliver continuous positive pressure gas from a ventilator to a user's nasal airway, which includes a front chamber section, a sealing structure, a ventilation component, and a positioning stabilizer. The front chamber section is bent towards one side of the user's face to form a main chamber and has at least one opening to install the ventilation component. The sealing structure has two shaped single-layer wall protrusions and communicates with the main chamber of the front chamber section. The ventilation component includes a noise reduction material, a ventilation component body, and a port cover. The positioning stabilizer is configured to be set on both ends of the front chamber section to provide a connection between components to secure the patient interface device. When in use, the exhaled waste gas is discharged into an external environment through the ventilation component.

### TECHNICAL FIELD

This disclosure is a patient interface device that seals with the user's nasal airway, which includes a front chamber section, a sealing structure, a ventilation component, and a positioning stabilizer. While ensuring the delivery of pressurized gas from the ventilator in a sealed manner, this disclosure also provides users with a comfortable wearing experience.

### BACKGROUND

With the increasing number of overweight and obese individuals worldwide, coupled with the escalating issues of an aging population, there has been a significant rise in patients suffering from sleep disordered breathing on a global scale. A 2022 research report projected that by 2024, approximately 1.15 billion people worldwide will suffer from sleep disordered breathing. Notably, this figure does not account for those under 30 or over 69 years old. Sleep disordered breathing is evolving into a grave societal concern, with its prevalence rate alarmingly high and continuing to rise. Generally, males have a higher incidence rate than females. Among the various types of sleep disordered breathing, Obstructive Sleep Apnea Syndrome (OSAS) stands out as the most common. Those with OSAS typically experience episodes of apena and hypoventilation during sleep often due to a narrowed pharynx, which is the primary characteristic of the condition. OSAS comes with a certain potential mortality rate and possible complications. The underlying pathogenesis of OSAS is characterized by chronic intermittent hypoxia and fragmented sleep, often accompanied by conditions such as hypoxemia and hypercapnia among a series of clinical syndromes due to physiological changes. Such complications can lead to damage across multiple body systems, which in turn might trigger severe health conditions like coronary heart disease, hypertension, diabetes, and other multi-organ, multi-system symptoms.

Continuous Positive Airway Pressure therapy (CPAP) is a common treatment method for OSAS. It is generally necessary for patients with moderate or severe sleep apnea to undergo ventilator-assisted treatment. CPAP therapy involves the coordinated use of various components, such as the hose, the elbow, the frame, and the patient interface device, and delivers pressurized gas from the ventilator to the patient's nasal or oral airway, either invasively or non-invasively. CPAP therapy is currently the most mainstream and recommended method for the treatment of OSAS. While components like the elbow or the frame play their part, the patient interface device commands special attention. The design of the patient interface device not only needs to ensure its compatibility with other components, a precise and stable sealing of the continuous positive pressure gas but also considers the user's comfort during facial contact. Among available options on the market, those patient interface devices solely for sealing the nasal airway are the smallest and lightest type among patient interface devices, with primary variations being invasive and non-invasive models.

With the growing market for the CPAP therapy components, a number of nasal masks and nasal pillows with noise reduction functions have emerged. Initially, the noise reduction function in these nasal masks or nasal pillows was achieved using cone-shaped ventilation holes similar to those in face masks, dispersing the airflow to achieve the effect of noise reduction. However, as users continued to use this type of nasal mask for more extended periods, more users demanded more from the nasal masks. In response, an invasive nasal mask with a detachable noise reduction ventilation component was introduced. This invasive design includes two protrusions that fit deep into the user's nostrils, thereby sealing the user's nasal airway. Nevertheless, this approach demands a high degree of precision in sealing the user's nasal airway, which leads to a more complex structure for a stable seal, further causing a lot of inconveniences for users during subsequent usage. Moreover, while existing noise reduction ventilation components on the market offer an improved method for expelling gas through corresponding structures, they fall short in terms of simplicity and ease of cleaning.

### SUMMARY

Therefore, this disclosure optimizes the sealing structure of nasal masks on the market. In this disclosure, a ventilation component that is both more streamlined and simpler to clean has been developed. Taking into account the continuous flow of pressurized air from the inlet to the user's nasal airway and the exhaled air passing through the nasal mask and out of the ventilation component, a patient interface device that is more efficient, convenient, and hygienic is provided. This design reduces the drawbacks found in existing devices on the market, bringing positive implications for both users and manufacturers.

This disclosure aims to introduce a novel patient interface device that has better sealing properties, offers enhanced cleanliness and drying, ensures more comfortable usage, and overcomes the limitations found in similar products of the existing methods. In this way, the disclosure offers broader application scenarios and spaces, along with more accessible methods (including structure, manufacturing, and production), configured to deliver the pressurized gas from a ventilator to the user's nasal airway in a sealed manner for the treatment of sleep disordered breathing

In one embodiment, a patient interface device is provided to deliver continuous positive pressure gas to a user's nasal airway in a sealed manner for the treatment of sleep disordered breathing, the patient interface device including at least some of the following elements or features.

A front chamber section includes a main chamber formed by a continuous wall and includes two entrance ends located on both sides, and the main chamber is curved towards a side nearer the user's face, to form a chamber structure with a certain curvature that doesn't make contact with the user.

A sealing structure, with a single-layer wall, is configured to be two shaped protrusions and communicates with the main chamber of the front chamber section. And at least a part of the sealing structure, when in use, contacts a portion of the user's nose and allows gas to be delivered through an exhaust end, to enter at least one nostril, thereby completing a pressurized gas delivery and sealing the user's nasal airway, with the two shaped protrusions being symmetrical to each other.

The front chamber section includes at least one opening, and the ventilation component is set on one of the openings of the front chamber section, with the ventilation component including:

A noise reduction material and a ventilation component body with two ports. The noise reduction material forms an inseparable whole with one port of the ventilation component body, the connection between the noise reduction material and the ventilation component body is seamless, and the noise reduction material first divides an airflow that exits from the front chamber section into smaller streams, before the airflow reaches an external environment through a mouth on one port of the ventilation component body away from the noise reduction material.

A positioning stabilizer is configured to be set on both ends of the front chamber section to provide a connection between components to secure the patient interface device to the user's face.

In one embodiment, the ventilation component includes a port cover connectable to the mouth on one port of the ventilation component body away from the noise reduction material, and a dispersed airflow from the ventilation component flows out to an external environment through the gap between a periphery of the port cover and the mouth of the ventilation component.

In one embodiment, the noise reduction material and the ventilation component body are made of a same material.

In one embodiment, the two protrusions are symmetrical themselves.

In one embodiment, the sealing structure has a non-uniform wall thickness.

In another embodiment, a patient interface device is provided in this disclosure to deliver continuous positive pressure gas to a user's nasal airway in a sealed manner for the treatment of sleep disordered breathing, the patient interface device including at least some of the following elements or features.

A front chamber section includes a main chamber formed by a continuous wall and includes two entrance ends located on both sides, and the main chamber is curved towards a side nearer the user's face, to form a chamber structure with a certain curvature that doesn't make contact with the user.

A sealing structure is configured to communicate with the main chamber of the front chamber section. And at least a part of the sealing structure, when in use, contacts at least a portion of the user's nose and allows the gas to be delivered through an exhaust end, to enter at least one nostril, to complete a pressurized gas delivery and to seal the user's nasal airway.

The front chamber section includes at least one opening, and the ventilation component is set on one of the openings of the front chamber section, with the ventilation component including:

A noise reduction material, a ventilation component body with two ports, and a port cover. The noise reduction material is integrally formed with one port of the ventilation component body, to make an inseparable whole, and the port cover is positioned on an other port of the ventilation component body away from the noise reduction material. The noise reduction material, through which an airflow that exits from the front chamber section is dispersed via its pores, is situated at a specific distance from the port cover, which allows the airflow, once dispersed by the noise reduction material to gather more gently before being discharged into an external environment, further reducing noise production.

There is a gap with a certain distance between a periphery of the port cover and a mouth of the ventilation component, configured to guide a direction of airflow through the noise reduction material and facilitate disassembly. The presence of the port cover results in different structures for an air inlet and an air outlet of the ventilation component.

A positioning stabilizer is configured to be set on both ends of the front chamber section to provide a connection between components to secure the patient interface device to the user's face.

In one embodiment, the noise reduction material and the ventilation component body are made of different materials.

In one embodiment, the positioning stabilizer is harder than the front chamber section.

In one embodiment, the port cover is connectable to the ventilation component body via a snap-fit connection.

In yet another embodiment, a patient interface device is provided in this disclosure to deliver continuous positive pressure gas to a user's nasal airway in a sealed manner for the treatment of sleep disordered breathing, the patient interface device including at least some of the following elements or features.

A front chamber section includes a main chamber formed by a continuous wall and includes two entrance ends located on both sides, and the main chamber is curved towards a side nearer the user's face, to form a chamber structure with a certain curvature that doesn't make contact with the user.

A sealing structure is configured to communicate with the main chamber of the front chamber section. And at least a part of the sealing structure, when in use, contacts at least a portion of the user's nose and allows the gas to be delivered through an exhaust end, to enter at least one nostril, to complete a pressurized gas delivery and to seal the user's nasal airway.

The front chamber section includes at least one opening, and the ventilation component is set on one of the openings of the front chamber section, with the ventilation component including:

A noise reduction material and a ventilation component body with two ports. The noise reduction material forms an inseparable whole with one port of the ventilation component body, and the connection between the noise reduction material and the ventilation component body is seamless. The noise reduction material first divides an airflow that exits from the front chamber section into smaller streams, before the airflow reaches an external environment through a mouth on one port of the ventilation component body away from the noise reduction material.

A positioning stabilizer is configured to be set on both ends of the front chamber section to provide a connection between components to secure the patient interface device to the user's face.

In one embodiment, the ventilation component includes a port cover connectable to the mouth on one port of the ventilation component body away from the noise reduction material, and a dispersed airflow from the ventilation component flows out to an external environment through a gap between a periphery of the port cover and the mouth of the ventilation component.

In one embodiment, the ventilation component is detachably connectable to the opening of the front chamber section.

In one embodiment, the positioning stabilizer extends in the direction of the openings at both ends of the front chamber section without bending itself.

In one embodiment, the sealing structure has a tendency to converge towards a point in the center.

In another embodiment, a patient interface device is provided in this disclosure to deliver continuous positive pressure gas to a user's nasal airway in a sealed manner for the treatment of sleep disordered breathing, the patient interface device including at least some of the following elements or features.

A front chamber section includes a main chamber formed by a continuous wall and includes two entrance ends located on both sides, and the main chamber is curved towards a side nearer the user's face, to form a chamber structure with a certain curvature that doesn't make contact with the user.

A sealing structure is configured to communicate with the main chamber of the front chamber section. At least a part of the sealing structure, when in use, contacts at least a portion of the user's nose and allows the gas to be delivered through an exhaust end, to enter at least one nostril, to complete a pressurized gas delivery and to seal the user's nasal airway.

The front chamber section includes at least one opening, and a ventilation component is set on one of the openings of the front chamber section, with the ventilation component including:

A noise reduction material and a ventilation component body with two ports. The noise reduction material forms an inseparable whole with one port of the ventilation component body, and the connection between the noise reduction material and the ventilation component body is seamless. The noise reduction material first divides an airflow that exits from the front chamber section into smaller streams, before the airflow reaches an external environment through a mouth on one port of the ventilation component body away from the noise reduction material.

A positioning stabilizer is configured to be set on both ends of the front chamber section to provide a connection between components to secure the patient interface device to the user's face.

An area of the opening of the front chamber section, where the ventilation component is installed, has a ratio of at least 1:9 to an outer wall area of the front chamber section, and the area of the opening of the front chamber section has a ratio of at least 5:1 to an area of the noise reduction material.

The noise reduction material has at least one of the following features:
1) the noise reduction material is made of fibrous material;
2) a thickness of the noise reduction material is at or between 0.2 to 5 mm;
3) one side of the noise reduction material has a surface area of at least 50 mm².

In one embodiment, the ventilation component has a port cover connectable to the mouth on one port of the ventilation component body away from the noise reduction material. And a dispersed airflow from the ventilation component flows out to an external environment through the gap between a periphery of the port cover and the mouth of the ventilation component.

In one embodiment, a periphery of the ventilation component includes a clamping structure that connects to the front chamber section.

In one embodiment, a shape of the noise reduction material is similar to the shape of the mouth connectable to the noise reduction material of the ventilation component body.

In one embodiment, the mouth connectable to the noise reduction material of the ventilation component body is not larger than the mouth of the ventilation component body connectable to the port cover.

In yet another embodiment, a patient interface device is provided in this disclosure to deliver continuous positive pressure gas to a user's nasal airway in a sealed manner for the treatment of sleep disordered breathing, the patient interface device including at least some of the following elements or features.

A front chamber section includes a main chamber formed by a continuous wall and includes two entrance ends located on both sides, and the main chamber is curved towards a side nearer the user's face, to form a chamber structure with a certain curvature that doesn't make contact with the user.

A sealing structure, with a single-layer wall, is configured to be two shaped protrusions and communicates with the main chamber of the front chamber section. And at least a part of the sealing structure, when in use, contacts a portion of the user's nose and allows the gas to be delivered through an exhaust end, to enter at least one nostril, thereby completing a pressurized gas delivery and sealing the user's nasal airway, with the two shaped protrusions being symmetrical to each other.

The front chamber section includes at least one opening, and a ventilation component is set on one of the openings of the front chamber section, with the ventilation component including:

A noise reduction material and a ventilation component body with two ports. The noise reduction material forms an inseparable whole with one port of the ventilation component body, and the connection between the noise reduction material and the ventilation component body is seamless. The noise reduction material first divides an airflow that exits from the front chamber section into smaller streams, before the airflow reaches an external environment through a mouth of the ventilation component body away from the noise reduction material.

A positioning stabilizer is configured to be set on both ends of the front chamber section to provide a connection between components to secure the patient interface device to the user's face.

An area of the opening of the front chamber section, where the ventilation component is installed, has a ratio of at least 1:9 to an outer wall area of the front chamber section, and the area of the opening of the front chamber section has a ratio of at least 5:1 to an area of the noise reduction material.

The noise reduction material has at least one of the following features:
1) the noise reduction material is one of foam-type or granular-type material;
2) a thickness of the noise reduction material is at or between 0.5 to 7 mm;
3) one side of the noise reduction material has a surface area of at least 50 mm².

In one embodiment, the ventilation component includes a port cover connectable to the mouth on one port of the ventilation component body away from the noise reduction material, a dispersed airflow from the ventilation component flows out to an external environment through a gap between a periphery of the port cover and the mouth of the ventilation component.

In one embodiment, for any two parallel cross-sections taken within the main chamber, the cross-sections are identical.

In one embodiment, the protrusions of the sealing structure are formed by extending in a normal direction from a specific location of the front chamber section. In one embodiment, each center of two exhaust ends on the sealing structure that contact the user's face, is equidistant from a center of the noise reduction material.

The benefits of the patient interface device provided by this disclosure can at least include:
1) The design has been optimized to be more airtight and comfortable to use. Existing patient interface devices on the market face challenges in ensuring airtight delivery of gas into the user's nasal airway. And some patient interface devices, in an effort to ensure structural stability, have a double-layer wall sealing structure. However, the use of this double-layer wall structure brings multiple issues to users. For instance, when the patient interface device contacts the user's nasal airway, it can cause discomfort, affecting subsequent treatment. The double-layer wall also reduces the size of the exhaust end opening, making it difficult for users to inhale the required amount of airflow. Moreover, the double-layer wall is prone to collecting dirt and contaminants, posing health risks for the user. There are also newer patient interface devices on the market that resemble a mini nasal mask, sealing from the bridge of the nose to the upper lip area. Yet, they still have shortcomings such as having a relatively large volume, being heavy, and being easily displaced during wear which can lead to air leakage. Recognizing these drawbacks, this disclosure improves the existing patient interface devices in response to the disadvantages of the sealing structure of the existing patient interface device. a. To provide users with a more comfortable experience, this disclosure has transformed the double-layer wall sealing structure of the current design into a single-layer wall structure. However, to ensure airtightness and the stability of the sealing structure, the size of the exhaust end opening of the patient interface device has been expanded through experimental testing. This disclosure also modifies the wall thickness to be non-uniform and changes the sealing structure to a symmetrical form. Such adjustments not only ensure the airtightness of the patient interface device but also address the shortcomings of the current devices and bring many advantages. For example, the single-layer wall has less impact on the user, reducing the potential discomfort or foreign body sensation within the nostrils, enhancing user comfort. It reduces instances where treatment is interrupted or users do not adhere to the treatment plan due to discomfort. The single-layer wall is also relatively lighter, decreasing the pressure sensation on the user's face for more comfortable wear. The design, which lacks a gap typically found between double walls, coupled with the enlarged opening at the exhaust end, ensures smoother breathing for users, preventing users from experiencing a sensation of insufficient airflow. To ensure that the single-layer wall structure is as stable as the double-layer wall structure, the non-uniform wall thickness of the sealing structure makes the relatively soft wall more comfortable when in contact with the user. The advantage of its symmetrical form is that when the exhaust end of the sealing structure enters the user's nostril, it exerts the same pressure thickness around the 360° structure of the nostril, providing more uniform pressure around the nostril. This makes users feel more comfortable during use, increasing their compliance with the treatment process. b) One form of the sealing structure of the patient interface device remains a single-layer wall with a symmetric, non-uniform thickness. This form seals the area from the user's nose tip to the upper lip region (including the nose tip). It only needs to ensure that the exhaust end size encompasses the user's nasal airway. Additionally, a spring structure is provided at the point where the exhaust end, which delivers continuous positive airway pressure, contacts the nose tip. This aids in adjusting to the varying nose shapes of different users. Therefore, the design of this patient interface device is adaptable, fitting a wide range of nostril sizes for enhanced airtightness. Unlike existing invasive nasal masks, this patient interface device does not enter the user's nostrils, reducing sensory disturbances and making it more suitable for the majority of people. This feature makes it particularly suitable for most users, especially those new to using devices for sleep apnea treatment, as they find adapting to wearing such devices easier. Also, compared to existing patient interface devices that seal the area from the user's nose bridge to the upper lip region, this device has a smaller volume which means it's easier for users to wear and easier to adjust when the nasal mask shifts. This design lowers the risk of gas leakage to some extent, benefiting users by making the device more portable and convenient when traveling, thereby facilitating the continuity of treatment.
2) This disclosure takes an innovative method for noise reduction. The ventilation component in this disclosure uniquely incorporates noise reduction material onto the ventilation component mouth to disperse the airflow. Moreover, the configured distance between the noise reduction material and the port cover ensures a gentler flow of gas to the external environment. In existing patient interface devices available on the market, the way to use the noise reduction material involves placing a piece of noise reduction cotton between both ports of the ventilation component. Although the airflow is dispersed by the noise reduction cotton, due to the limited space between the cotton and both ports, the airflow becomes turbulent at both the air inlet and air outlet of the ventilation component, which is not conducive to the use by the user. Additionally, positioning the noise reduction cotton in a non-detachable manner between both ports of the ventilation component makes it challenging for users to clean. Innovatively, this disclosure positions the noise reduction material, including a noise reduction mesh set at the air inlet of the ventilation component, establishing a certain gap between the noise reduction material and the second port (which is also the port cover's location) of the ventilation component. As gas flows from the front chamber to the external environment through the ventilation component, it first disperses through the noise reduction material, then a turbulent airflow becomes a gentler flow due to the gap between the noise reduction material and the port cover, eventually exiting to the external environment through the gap between the port cover and the ventilation component. Compared to existing patient interface devices, such a process results in more apparent noise reduction and allows the airflow to be directed to the user in a non-frontal manner, reducing the possibility of airflow blowing onto the user's face. In summary, the ventilation component of the disclosure outperforms the current ventilation component of the patient interface in several aspects.
3) The design is modular, which expands the range of user choices. This disclosure introduces a modular design for the ventilation component. Compared to existing designs, the ventilation component of this disclosure allows for the replacement of the port cover and noise reduction material. Users can choose and replace the appropriate noise reduction material and user-friendly type of port cover to fit the ventilation component body in accordance with their preferences and needs. In this way, users are provided with diverse options. a. The port cover of the ventilation component in this disclosure is detachably connectable to the ventilation component. Furthermore, the noise reduction material is internally formed with the ventilation component body in a non-detachable manner, rather than being placed in the middle part of the component. As a result, the presence or absence of a port cover does not cause the noise reduction material to fall out due to a lack of obstruction, and it does not essentially impact the noise reduction effect. The primary role of the port cover in this design is to leave a gap between the ventilation component and the noise reduction material, turning the turbulent airflow that passes through the noise reduction material into a gentler flow to the external environment. However, even without the port cover, the airflow from the front chamber will still be dispersed through the noise reduction material, achieving the noise reduction effect. While having a port cover offers certain advantages, the port cover isn't the primary component providing noise reduction. Therefore, users can choose whether or not to use the port cover, and still achieve noise reduction. This provides an option for users to experience noise reduction without using the port cover at their own acceptance. b. In scenarios where the port cover is used, the detachable port cover allows users to choose their preferred port cover and its connection methods, which include options like a fully detachable port cover or a flip port cover. Different port covers and connection methods have their pros and cons. Users can choose the fully detachable port cover for detailed cleaning of the interior of the ventilation component, or opt for the flip port cover, which is more convenient for users to snap-fit it in place and is less likely to fall off, avoiding situations where the port cover cannot be found. Both types of port covers are more conducive to cleaning and drying the noise reduction material compared to closed port covers. c. This disclosure has a replaceable design for the noise reduction material. Since the noise reduction material is integrated with the ventilation component body in a non-detachable manner, the mouth on one port of the ventilation component has a detachable port cover. The ventilation component, consisting of only two parts, facilitates a modular design. Therefore, this design allows the connection of the ventilation component body with different types or forms of noise reduction material to the same port cover. Users can choose ventilation components with different types or forms of noise reduction materials to meet their diverse needs for ventilation. This approach adds flexibility to the entire patient interface device product. The replaceable modules can cater to users' varying needs and preferences, reducing the complexity of the ventilation component.
4) This disclosure ensures a more thorough and hygienic cleaning. As the product comes in direct contact with the user's face and communicates with the user's airway, maintaining the hygiene of the respiratory pathway is needed for the user's health, which effectively reduces the risk of infection intake. a. The port cover on the outlet side of the patient interface device in the existing market is usually a non-detachable whole with the ventilation component, and the noise reduction material is placed inside the ventilation component on the inner side of the port cover, which is very unfavorable to the user's cleaning of the ventilation component. When users clean the internal noise reduction material (i.e., noise reduction cotton) or after prolonged use when moisture accumulates in the noise reduction cotton, the inseparability of the port cover means that only small gaps exist for drying the noise reduction cotton. This can easily lead to incomplete drying and then bacterial growth. However, this disclosure makes the port cover detachable, enabling a more thorough and cleaner cleaning by directly accessing the noise reduction material by opening the port cover. The port cover can also be opened for drying and ventilation, offering higher hygiene standards compared to existing products, which effectively reduces the risk of infection intake. Furthermore, the ventilation component of this disclosure has two layers: the noise reduction material on one port, the port cover on the other port.

This design is overall more streamlined than the three-layer form available on the market with the noise reduction material in the middle part of the ventilation component, which facilitates the cleaning and disinfection of both the entire ventilation component and its details, leaving no narrow gaps uncleaned. b. In addition, the noise reduction material used in this disclosure can be a noise reduction mesh. The noise reduction cotton used in the current market is difficult to clean and dry due to the non-openable port cover, and it easily absorbs exhaled moisture, leading to the growth of bacteria and mold. In contrast, the noise reduction mesh material doesn't have these issues. It does not absorb moisture, and the port cover can be opened, allowing the noise reduction mesh to be scrubbed repeatedly with a brush, so that the ventilation component is more durable. The noise reduction mesh used in this disclosure can withstand repeated cleaning without getting damaged, offering more protection for the user's health. c. The double-layer wall technology available on the current market tends to accumulate dirt and is not easy to clean. Over time, users can be easily infected by bacteria and other microbes, posing a risk of illness. This disclosure, through the aforementioned foundational improvements, changes the sealing structure from the double-layer wall on the market to a single-layer wall structure. Not only does this present advantages in usage, but in terms of cleaning, compared to multi-layer wall structures, it is more straightforward. Users can clean the sealing structure more easily, without the internal crevices or hard-to-reach areas found in multi-layer wall structures, making cleaning quicker and more effective.
5) This disclosure has reduced processes, a lower cost, a decreased defect rate, and is environmentally friendly. The patient interface device in this disclosure differs from current products in both the sealing structure and the ventilation component. With the innovative improvements of this disclosure, apart from the aforementioned advantages, the product also has fewer production processes, lower manufacturing costs and a lower defect rate. Moreover, the improved technology results in a reduced material usage, benefiting the environment. a. The improvements mentioned above in this disclosure make the sealing structure change from a double wall in the market to a single-layer wall structure. From a manufacturing standpoint, the single-layer wall design is more cost-effective than the multi-layer wall, as the latter requires more material and a relatively more complex production process and mold cost. In comparison, the production process of the single-layer wall is more economical and efficient. This approach also conserves raw materials used in manufacturing the patient interface. By saving on material usage, there is a reduced demand for plastics, polyester, silicone, etc., leading to decreased carbon emissions. Continuing to adopt this green design approach will have a positive impact on a larger scale, taking a step nearer towards carbon neutrality. b. In this disclosure, the noise reduction material and the mouth of the ventilation component body where the airflow enters the ventilation component body from the front chamber are integrally formed, different from the current patient interface devices where components and noise reduction materials are produced separately and then assembled. And this design simplifies the production process by using the method of manufacturing a single, unified product without the need for assembly, which is faster compared to the manufacturing and assembling steps of existing products, thereby reducing production time, labor costs, production processes, and equipment requirements. The integral fabrication also enhances the structural strength of the ventilation component to a certain extent. Because parts are made and connected in one operation, the possibility of assembly errors is decreased. This method enhances product quality and reliability, making the product more durable, further realizing an environmentally friendly design.

### BRIEF DESCRIPTION OF THE DRAWINGS

FIG. 1 is a three-dimensional schematic diagram of a patient interface device in accordance with an example embodiment;

FIG. 2 is a three-dimensional schematic diagram of a ventilation component of a patient interface device in accordance with an example embodiment;

FIG. 3 is an exploded schematic diagram of a patient interface device in accordance with an example embodiment;

FIG. 4 is a front view and a cross-sectional schematic diagram along the A-A axis of a patient interface device in accordance with an example embodiment;

FIG. 5 is a front view and a cross-sectional schematic diagram along the B-B axis of a patient interface device in accordance with an example embodiment;

FIG. 6 is a front view and a cross-sectional schematic diagram along the C-C axis of a ventilation component in accordance with an example embodiment;

FIG. 7 is a schematic diagram illustrating the airflow path through a noise reduction mesh as it exits the ventilation component in accordance with an example embodiment;

FIG. 8 is a schematic diagram illustrating the airflow path through a noise reduction cotton as it exits the ventilation component in accordance with an example embodiment;

FIG. 9 is a schematic diagram illustrating the airflow entering and exiting a patient interface device in accordance with an example embodiment;

FIG. 10 is a schematic diagram illustrating the positional relationship between the sealing structure and the ventilation component in a patient interface device in accordance with an example embodiment;

FIG. 11 is a cross-sectional view of the wall thickness of a sealing structure in a patient interface device in accordance with an example embodiment;

FIG. 12 a cross-sectional view of the wall thickness of a sealing structure in a patient interface device in accordance with another example embodiment;

FIG. 13 is a schematic diagram illustrating the sealing structure as a symmetrical configuration and its mutually symmetrical arrangement in accordance with an example embodiment;

FIG. 14 is a schematic diagram for cleaning the ventilation component in accordance with an example embodiment;

FIG. 15 is a schematic diagram for cleaning the sealing structure in accordance with an example embodiment;

FIG. 16 is a cross-sectional view of a mold of the patient interface device in accordance with Embodiment 1;

FIG. 17 is a three-dimensional schematic diagram of a patient interface device with different sealing structures in accordance with Embodiment 2;

FIG. 18 is a three-dimensional schematic diagram of the patient interface device in accordance with another embodiment in Embodiment 2;

FIG. 19 is a three-dimensional schematic diagram of the patient interface device using different noise reduction materials in accordance with Embodiment 3;

FIG. 20 is a three-dimensional schematic diagram of the patient interface device without a port cover in accordance with Embodiment 4;

FIG. 21 is a three-dimensional schematic diagram of the patient interface device with different forms of a port cover in accordance with Embodiment 5.

### DETAILED DESCRIPTION

To facilitate an understanding of the disclosure, a more comprehensive description will be given below with reference to the relevant figures. The figures illustrate typical embodiments of the disclosure. However, the disclosure can be implemented in many different forms and is not limited to the embodiments described herein. Instead, these embodiments are provided to make the disclosure more thorough and comprehensive.

Unless otherwise defined, all technical and scientific terms used herein have the same meaning as commonly understood by those skilled in the technical field to which the disclosure belongs. The terms used in the description of the disclosure herein are used only for the purpose of describing specific embodiments and are not intended to limit the disclosure.

This disclosure addresses the problems of existing patient interface devices used to deliver gas to the user's nasal airway, which are difficult to clean, not conducive to the user's comfort, overly complex in ventilation components, difficult to produce, and costly. A patient interface device with a simpler structure and a comfortable wearing experience for the user is provided in this disclosure. The patient interface device of this disclosure not only optimizes the various disadvantages of the existing design but also provides several different structures of patient interface devices for users to choose according to their needs and preferences. This disclosure represents a better technical innovation for users, producers, and the market. The reduction of material waste by this disclosure is also a sustainable, environmentally friendly design.

Detailed embodiments are presented below to elucidate the structures of a patient interface device.

### Embodiment 1

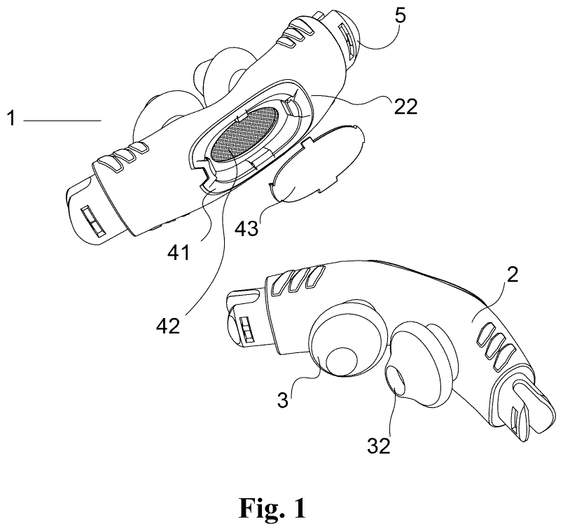

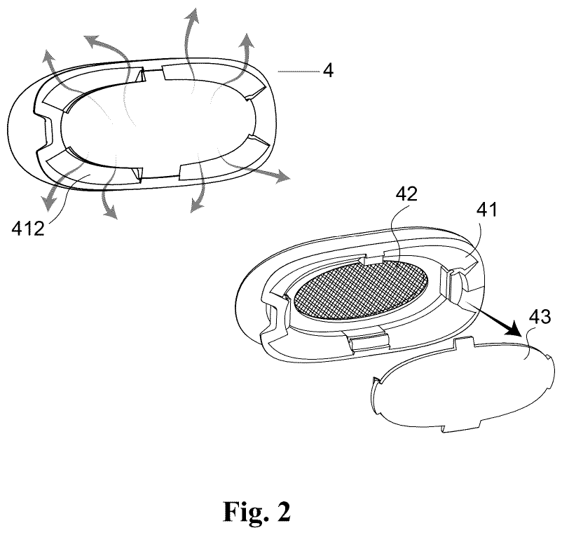

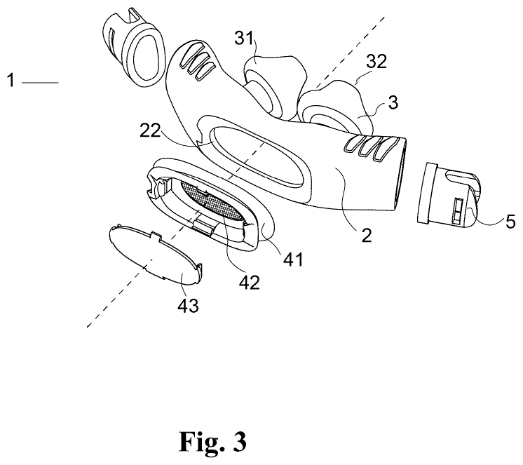

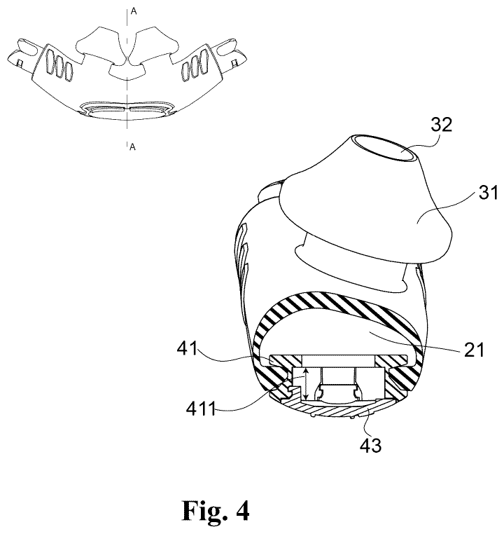

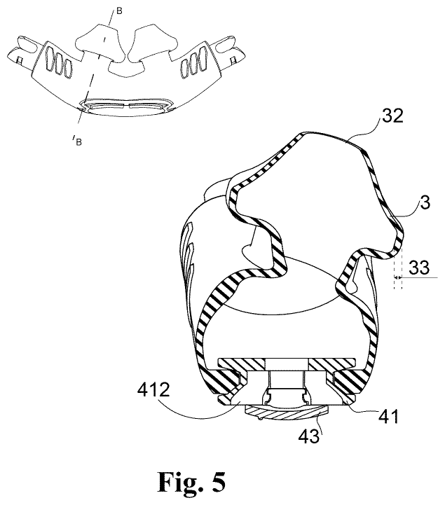

This embodiment provides a patient interface device 1 configured for delivering continuous positive pressure gas to the user's nasal airway in a sealed manner for the treatment of sleep disordered breathing. This embodiment presents a three-dimensional schematic diagram, an exploded view, a front view, a schematic cross-sectional view, an airflow path diagram, a positional relationship diagram, a diagram for demonstration of the cleaning, and a cross-sectional diagram of a mold of the patient interface device 1, as shown in FIGS. 1-16. The patient interface device 1 shown in the embodiment includes a front chamber section 2, a ventilation component 4, a sealing structure 3, and a positioning stabilizer 5. It is configured to seal the area below the user's nose tip to the upper lip and to deliver continuous positive pressure gas to the user's nasal airway for the treatment of obstructive sleep apnea symptoms.

The patient interface device 1 has a front chamber section 2, which has a main chamber 21 made of a continuous wall and entrance ends of the front chamber section 2 on both sides. The main chamber 21 bends towards the side nearer the user's face, forming a chamber structure with a certain curvature that doesn't make contact with the user. When the user uses this patient interface device 1 for treatment, the continuous pressurized gas output from the ventilator first enters from the entrance ends of the front chamber section 2 on both sides of the patient interface device 1, then stores part of the gas in the main chamber 21. Within the main chamber 21, any two parallel cross-sections taken can be either identical or different. The front chamber section 2 is made of a flexible material with a hardness of 30 A to 70 A on the Shore A scale, such as silicone, rubber, or elastic plastic. Alternatively, the front chamber section 2 can also be composed of rigid plastic materials, such as high-density polyethylene, polypropylene, polycarbonate, polystyrene, and others.

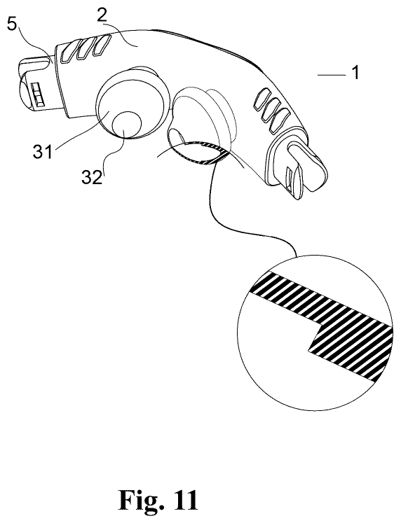

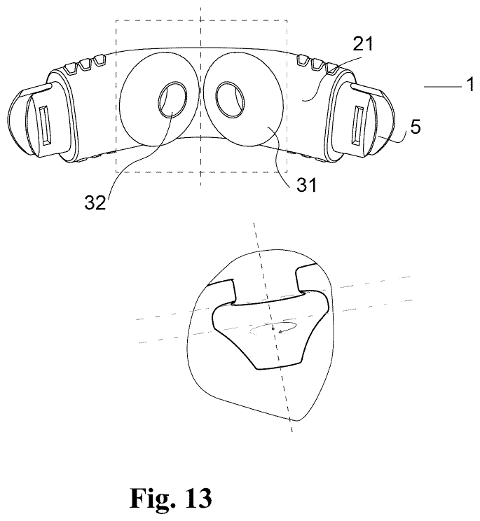

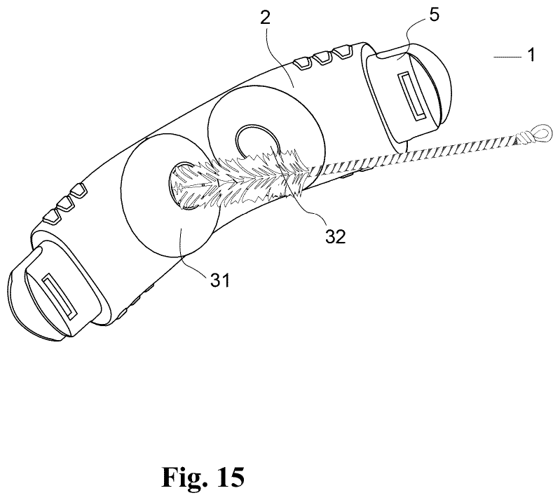

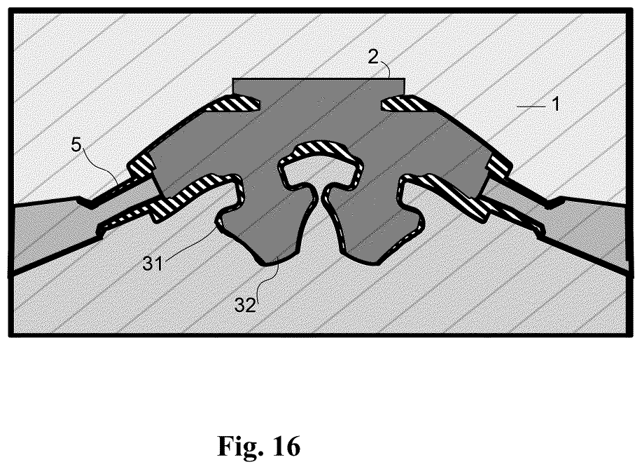

The patient interface device 1 also includes a sealing structure 3 with a single-layer wall, configured to be two shaped protrusions 31 that communicate with the main chamber 21 of the front chamber section 2. When in use, at least part of the sealing structure 3 contacts at least a portion of the user's nose, allowing gas to be delivered through an exhaust end, entering at least one nostril, completing a pressurized gas delivery and sealing the user's nasal airway. The two shaped protrusions 31 are symmetrical to each other (as shown in FIG. 13). The sealing structure 3 has a tendency to converge towards a point in the center, which means the axes of the protrusions 31 extend and intersect at a single point. Since there are many differences in the height of users' noses and the size of their nostrils, existing patient interface devices 1, to ensure the sealing effect of the gas flow, have a double-layer wall in the sealing structure 3. Although this design improves the sealing effect to some extent, the double-layer wall also brings more disadvantages to the patient interface device 1. This disclosure, through testing and design, changed the sealing structure 3 to a single-layer wall and also changed the structure of the ventilation component 4. The single-layer wall design achieves the sealing function of the double-layer wall without needing the asymmetrical design of the sealing structure found in existing market devices to improve the sealing effect. Therefore, in this disclosure, the two protrusions 31 are symmetrical themselves (as shown in FIG. 13). Considering the comfort of the user's nose when wearing, each center of two exhaust ends on the sealing structure 3 that contact the user's face, is equidistant from the center of the noise reduction material 42. That is to say, the exhaust ends of the sealing structure 3 are symmetrical to each other. The protrusions 31 of the sealing structure 3 are formed by extending in the normal direction from a specific location (which corresponds to the position of the user's nostrils) of the front chamber section 2 until they contact and seal with the user's nose to transmit pressurized gas. The double-layer sealing structure in the existing market also serves a supporting function. While this disclosure, replacing the double-layer wall with a single-layer one, enlarges the exhaust end opening for the flow of pressurized gas, allowing for smoother inhalation and exhalation by the user, for the stability of its shape, the sealing structure 3 has a non-uniform wall thickness, having at least one thin area and a non-thin area thicker than the thin area as a support region, specifically, certain areas of the sealing structure have thickened walls as a support region while other parts are a thin region (as shown in FIG. 11). In addition, the mold development costs of the single-layer wall are relatively lower and easier to achieve compared to the double-layer wall (as shown in FIG. 16), as its molds are simpler without the need to consider the difficulty of demolding due to the two layers at the exhaust end of the double-layer wall. And the single-layer patient interface device is easier to clean (as shown in FIG. 15). The sealing structure 3 in this disclosure is configured in such a way that it does not position or press against the user's nasal columella when sealing with the user's face, making the user's treatment process more comfortable without the sensation of a foreign object touching the nose.

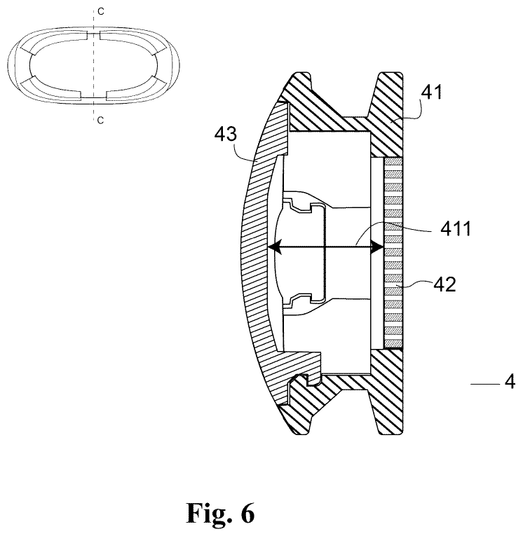

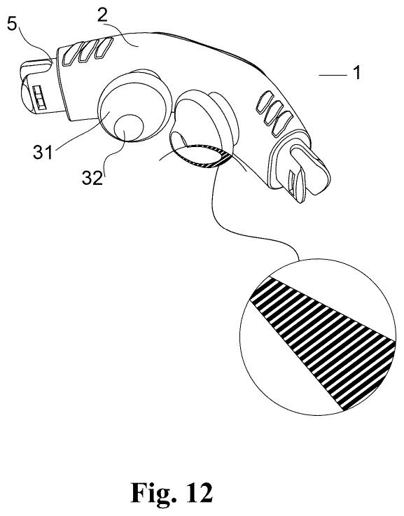

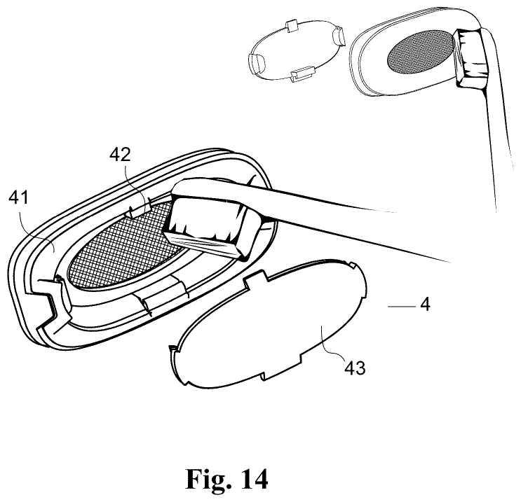

The patient interface device 1 includes a front chamber section 2 that has at least one opening. A ventilation component 4 is located on one of the openings of the front chamber section 2. The ventilation component 4 includes a noise reduction material 42 and a ventilation component body 41 with two ports. The noise reduction material 42 forms an inseparable whole with the mouth on one port of the ventilation component body 41, and there is a seamless connection between the noise reduction material 42 and the ventilation component body 41. For the sake of ease in manufacturing and aesthetic considerations, the shape of the noise reduction material 42 is similar to the shape of the mouth in the ventilation component body 41, where it connects with the noise reduction material 42. In other words, if the shape of the ventilation component body 41 is square, then the noise reduction material 42 should also be square, or a similar rounded rectangle, and not a significantly different shape like a triangle. When the airflow is expelled from the front chamber section 2, it is first divided into smaller streams by the noise reduction material 42, and then it flows out into the external environment through the port of the ventilation component body 41 that is away from the noise reduction material 42. The periphery of the ventilation component 4 can have a clamping structure (as shown in FIG. 6, an indentation is set in the middle of the ventilation component 4, forming a groove that accommodates the corresponding protrusion of the front chamber section 2) for a detachable connection with the front chamber section 2, or the ventilation component 4 can be configured for a non-detachable connection with the front chamber section 2. In one embodiment, the ventilation component 4 has a port cover 43 connectable to the mouth of the ventilation component body 41 that is away from the noise reduction material 42. There is a gap (more than 1 mm, which ensures that the user can exhale waste gas normally) between the periphery of the port cover 43 and the mouth of the ventilation component 41, which is configured to guide the direction of airflow through the noise reduction material 42 and to facilitate easy disassembly. The presence of the port cover 43 results in distinct structural differences between the air inlet (where the exhaled waste gas of the user enters into the ventilation component 4) and the air outlet of the ventilation component 4. The dispersed airflow flows out into the external environment through the gap between the periphery of the port cover 43 and the mouth of the ventilation component 4. That is, when the exhaled waste gas enters the ventilation component 4, it enters through the pores between the noise reduction material 42, and when the exhaled waste gas exits the ventilation component 4, it exits to the external environment through the mouth. The port cover 43 is positioned a certain distance away from the noise reduction material 42 (more than 3.5 mm). This distance is configured to gather the dispersed airflow from the noise reduction material 42 and release it gently into the external environment (with gas flow rates ranging between 0.1 to 6 liters per second) to further reduce noise production. To prevent the gas flowing exiting the noise reduction material 42 from directly entering into the external environment, the mouth of the ventilation component body 41 connectable to the noise reduction material 42 is configured to be no larger than the mouth of the ventilation component body 41 connectable to the port cover 43. The port cover 43 is connectable to the ventilation component body 41 through a snap-fit or another method, forming a detachable or semi-detachable connection. These distinguish from and are advantageous over existing ventilation components, as the ventilation component 4 has only two layers, making it structurally simpler and easier to clean compared to the three layers of existing components (as shown in FIG. 12 and FIG. 14). The noise reduction material 42 and the ventilation component body 41 can be made of the same or different materials, providing a more diverse selection for the users. For supporting the noise reduction material 42, the ventilation component body 41 is provided as a rigid material including one or more materials such as high-density polyethylene, polypropylene, polycarbonate, polystyrene, and the like. To ensure smooth airflow in the patient interface device 1, the following data have been restricted in this disclosure: The area of the opening of the front chamber section 2 where the ventilation component 4 is installed has at least a 1:9 ratio to the outer wall area of the front chamber section 2; and the area of the opening of the front chamber section 2 has at least a 5:1 ratio to the area of the noise reduction material 42. In one embodiment, the noise reduction material 42 has at least one of the following features: 1) the noise reduction material is made of fibrous material (such as polyester fiber cotton or glass fiber cotton); 2) the thickness of the noise reduction material is at or between 0.2 to 5 mm; 3) one side of the noise reduction material has a surface area of at least 50 mm². Additionally, the patient interface device 1 in this embodiment has a positioning stabilizer 5, which is configured to provide a connection between components at both ends of the front chamber section 2 to secure the patient interface device 1 to the user's face. The positioning stabilizer 5 is configured to extend in the direction of the opening at both ends of the front chamber section 2 without bending itself, which means the positioning stabilizer 5 is straight, extending linearly along the direction of the openings at both ends of the front chamber section 2. Besides, the positioning stabilizer 5 is harder than the front chamber section 2. The positioning stabilizer 5 is used to join with other components for treating sleep disordered breathing and can be made from either rigid or non-rigid materials. The material composition of the positioning stabilizer 5 can include silicone, rubber, elastic plastic, or rigid materials such as high-density polyethylene, polypropylene, polycarbonate, or polystyrene.

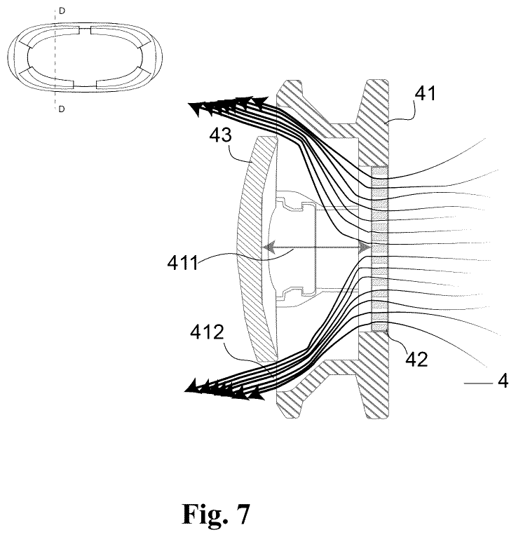

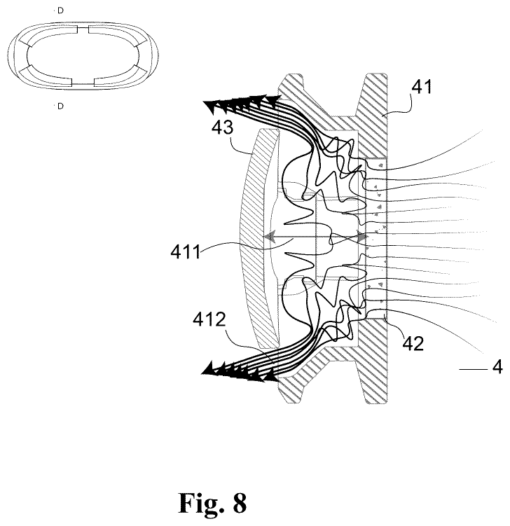

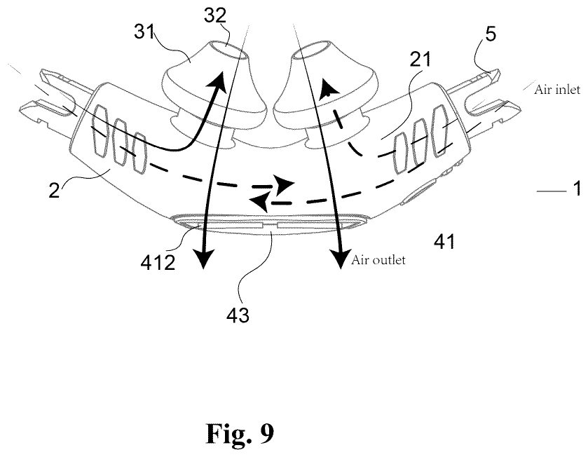

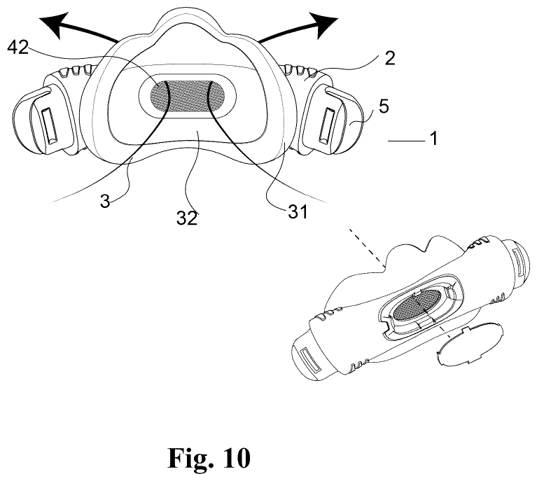

During use, the airflow first enters the patient interface device 1 from the entrance ends of the front chamber section 2 on both sides, flowing through the chamber that bends towards the side nearer the user's face. It then flows into the user's nasal airway through the sealing structure 3. In the exhalation process, the waste gas exhaled by the user first passes through the sealing structure 3 and is then expelled into the external environment in an almost straight line via the ventilation component 4 (as shown in FIG. 9). Specifically, the waste gas is first dispersed and noise-reduced by the noise reduction material 42 within the ventilation component 4, then passes through the chamber between the noise reduction material 42 and the opposite mouth on the other port of the ventilation component body 41, where the turbulent airflow gradually slows down. In the presence of the port cover 43, the waste gas finally flows out into the external environment through the gap between the periphery of the port cover 43 and the mouth on one port of the ventilation component body 41 away from the noise reduction material (as shown in FIG. 7 and FIG. 8). The slowed airflow, through the chamber between the noise reduction material 42 and the mouth on the other port of the ventilation component body 41, helps to reduce the likelihood of airflow blowing onto the user's face, thereby further reducing noise and enhancing the noise reduction effect. The ventilation component 4 is set on the central opening of the front chamber section 2, which means that when the positive pressure airflow enters through the entrance ends on both sides, the ventilation component 4 is on the path through which the airflow passes, while the direction of the mouths on the ventilation component 4 is not parallel to the direction of the airflow. As a result, there is little or no expulsion of the positive pressure airflow into the external environment through the ventilation component 4 (as illustrated in FIG. 9). The position of the ventilation component 4 is exactly opposite the sealing structure 3, with their central points both located on the symmetry axis of the patient interface device 1. Thus, during exhalation, the waste gas can exit the chamber through a shorter path and precise position, facilitating ventilation for the user (as shown in FIG. 10).

In another embodiment, the wall thickness of the sealing structure 3 of the patient interface device 1 can also be uniformly variable, with a thicker area at the bottom serving as a support region and a thinner area above as the thin region (as shown in FIG. 12).

### Embodiment 2

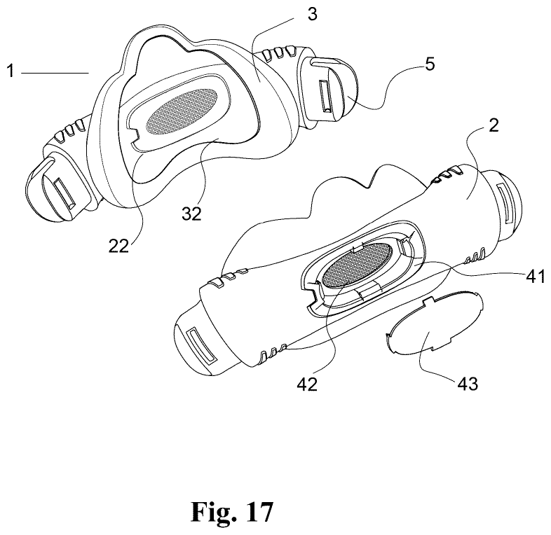

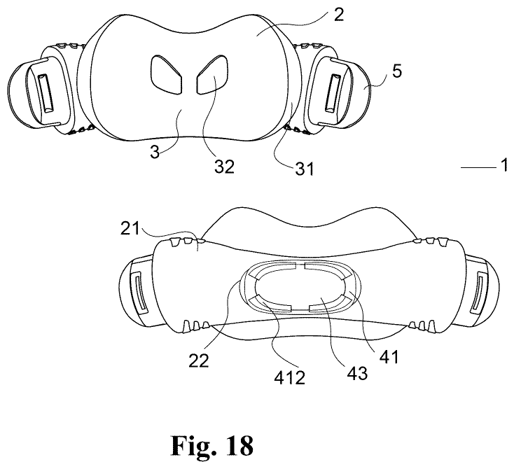

This embodiment provides a patient interface device 1 that seals with the user's nasal airway, as illustrated in FIG. 17 and FIG. 18. A three-dimensional schematic diagram of the patient interface device 1 is provided in this embodiment. As shown in FIGS. 17, 18, the differences in the patient interface device 1 of this embodiment from that of Embodiment 1, are that the sealing structure 3 of the patient interface device 1 herein is configured to seal the area from the user's nose tip to the upper lip. The front chamber section 2 and the sealing structure 3 have different forms than those in Embodiment 1 and are configured to non-invasively surround the area at least including the nose tip, the first nostril, and the second nostril of the user, to make contact with the area around the nostrils. Since the size and height of the noses of different users always vary, the patient interface device 1 in this embodiment only needs to ensure that the exhaust end is large enough to enclose the user's nasal airway, thus solving the problem of air leakage which occurs when the device does not perfectly fit the user's nose. This not only achieves a better seal but also reduces the potential discomfort or foreign body sensation inside the nostrils that users might feel, thereby improving the comfort of use.

In another embodiment, the front chamber section 2 of the patient interface device 1 is made of a rigid material, and the sealing structure 3 is configured non-invasively to surround the user's face without including the nose tip area (as shown in FIG. 18).

### Embodiment 3

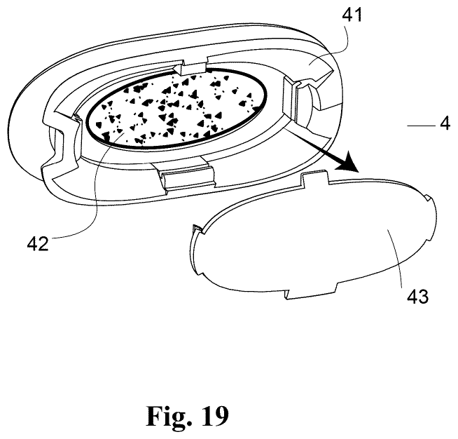

This embodiment presents a patient interface device 1 that seals with the user's nasal airway, as shown in FIG. 19. It provides a diagram of the airflow path exiting the ventilation component 4 and a three-dimensional schematic of the patient interface device 1. In this embodiment, as shown in FIG. 8 and FIG. 19, the difference from the patient interface device 1 in Embodiment 1 is that this embodiment limits the properties of the noise reduction material 42. As for the ventilation component 4 of the patient interface device 1 in this embodiment, the ventilation component body 41 and the noise reduction material 42 form a seamless and inseparable connection. The noise reduction material 42 has at least one of the following features: 1) the noise reduction material 42 is either a foam-type or granular-type material (such as polyurethane foam plastic, foam metal, polyurethane foam); 2) the thickness of the noise reduction material 42 is at or between 0.5 to 7 mm; 3) one side of the noise reduction material 42 has a surface area of at least 50 mm². The patient interface device 1 can have different noise reduction materials 42 appropriately sized to fit onto one port of the ventilation component body 41, which is away from the port cover 43, and connectable to the ventilation component body 41. The noise reduction material 42 is connectable to the ventilation component body 41 either by injection molding, ultrasonic welding, thermal pressing, etc., or using adhesives such as glue or tape, or by a snap-fit, a knob, a clip, or with the help of a third component (a strap, a clip), ultimately forming an inseparable whole. The manner in which several noise reduction materials 42 are connectable to the ventilation component body 41 to form different ventilation components 4 provides users with more diverse options, allowing them to choose a ventilation component 4 that is more suitable for their own use, facilitating the continuity of treatment for sleep disordered breathing.

### Embodiment 4

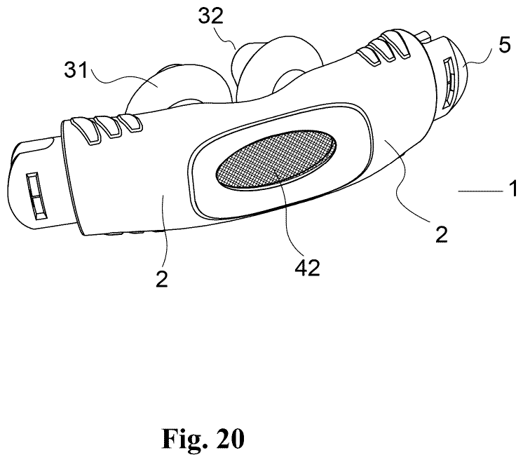

This embodiment provides a patient interface device 1 that seals with the user's nasal airway, as shown in FIG. 20. A three-dimensional schematic of the patient interface device 1 is provided in this embodiment. In the embodiment illustrated by FIG. 20, the patient interface device 1 differs from that of Embodiment 1 in that the ventilation component 4 of the patient interface device 1 does not have a port cover 43. In the ventilation component 4 of this disclosure, the noise reduction material 42 and the ventilation component body 41 are integrally formed as an inseparable connection, rather than being positioned in the middle part of the ventilation component 4. As such, the absence of the port cover 43 does not cause the noise reduction material 42 to fall out due to a lack of obstruction and it does not affect the use of the patient interface device 1. Moreover, the noise reduction effect of the ventilation component 4 is primarily achieved by the noise reduction material 42, not the port cover 43. Whether having a port cover 43 or not does not impact the noise reduction effect over a wide area. Even without a port cover 43, the airflow from the front chamber section 2 still passes through the noise reduction material 42 for dispersal of the airflow which achieves a noise reduction effect. The exhalation process of the user remains the same, where the waste gas from the user is first directed through the sealing structure 3, then passes through the ventilation component 4 in an almost straight line, before being expelled into the external environment. Specifically, the waste gas first undergoes dispersion for noise reduction by the noise reduction material 42 within the ventilation component 4, then slows down after passing through the chamber between the noise reduction material 42 and the mouth at one port of the ventilation component body 41, and is finally directly released into the external environment.

### Embodiment 5

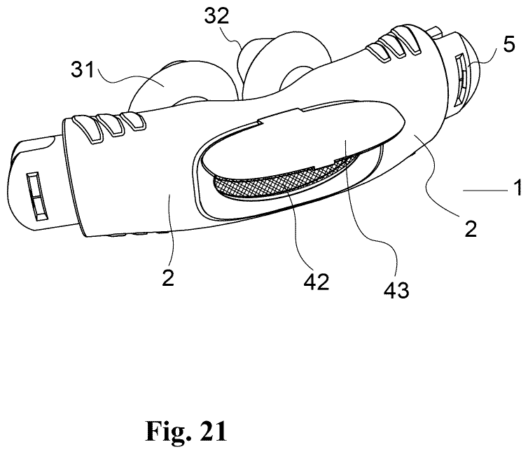

This embodiment provides a patient interface device 1 that seals with the user's nasal airway, as illustrated in FIG. 21. A three-dimensional schematic of the patient interface device 1 is presented in this embodiment. As shown in FIG. 21, the difference between the patient interface device 1 of this embodiment and that of Embodiment 1, is that the port cover 43 of the ventilation component 4 is configured to be a semi-detachable flip cover. This disclosure, as shown in the embodiment, allows users to choose their preferred port cover 43 and its connection methods. Different port covers 43 and their connection methods have their advantages and disadvantages. The semi-detachable flip cover design facilitates the user's cleaning and drying of the noise reduction material 42 and the interior details of the ventilation component 4 compared to a sealed port cover 43. While it may not provide the convenience of cleaning like a fully detachable port cover 43, its semi-detachable nature reduces the risk of misplacement or loss, a common issue with fully detachable covers that need removal of the port cover 43 for every cleaning session. The port cover 43 is even more difficult to be found due to its smaller size. Therefore, the ventilation component 4 is provided with a semi-detachable flip port cover 43 to avoid the problem of losing the cover while still offering a range of options for user preference.

The benefits of the patient interface device provided by this disclosure can at least include:
1) The design has been optimized to be more airtight and comfortable to use. Existing patient interface devices on the market face challenges in ensuring airtight delivery of gas into the user's nasal airway. And some patient interface devices, in an effort to ensure structural stability, have a double-layer wall sealing structure. However, the use of this double-layer wall structure brings multiple issues to users. For instance, when the patient interface device contacts the user's nasal airway, it can cause discomfort, affecting subsequent treatment. The double-layer wall also reduces the size of the exhaust end opening, making it difficult for users to inhale the required amount of airflow. Moreover, the double-layer wall is prone to collecting dirt and contaminants, posing health risks for the user. There are also newer patient interface devices on the market that resemble a mini nasal mask, sealing from the bridge of the nose to the upper lip area. Yet, they still have shortcomings such as having a relatively large volume, being heavy, and being easily displaced during wear which can lead to air leakage. Recognizing these drawbacks, this disclosure improves the existing patient interface devices in response to the disadvantages of the sealing structure of the existing patient interface device. a. To provide users with a more comfortable experience, this disclosure has transformed the double-layer wall sealing structure of the current design into a single-layer wall structure. However, to ensure airtightness and the stability of the sealing structure, the size of the exhaust end opening of the patient interface device has been expanded through experimental testing. This disclosure also modifies the wall thickness to be non-uniform and changes the sealing structure to a symmetrical form. Such adjustments not only ensure the airtightness of the patient interface device but also address the shortcomings of the current devices and bring many advantages. For example, the single-layer wall has less impact on the user, reducing the potential discomfort or foreign body sensation within the nostrils, enhancing user comfort. It reduces instances where treatment is interrupted or users do not adhere to the treatment plan due to discomfort. The single-layer wall is also relatively lighter, decreasing the pressure sensation on the user's face for more comfortable wear. The design, which lacks a gap typically found between double walls, coupled with the enlarged opening at the exhaust end, ensures smoother breathing for users, preventing users from experiencing a sensation of insufficient airflow. To ensure that the single-layer wall structure is as stable as the double-layer wall structure, the non-uniform wall thickness of the sealing structure makes the relatively soft wall more comfortable when in contact with the user. The advantage of its symmetrical form is that when the exhaust end of the sealing structure enters the user's nostril, it exerts the same pressure thickness around the 360° structure of the nostril, providing more uniform pressure around the nostril. This makes users feel more comfortable during use, increasing their compliance with the treatment process. b) One form of the sealing structure of the patient interface device remains a single-layer wall with a symmetric, non-uniform thickness. This form seals the area from the user's nose tip to the upper lip region (including the nose tip). It only needs to ensure that the exhaust end size encompasses the user's nasal airway. Additionally, a spring structure is provided at the point where the exhaust end, which delivers continuous positive airway pressure, contacts the nose tip. This aids in adjusting to the varying nose shapes of different users. Therefore, the design of this patient interface device is adaptable, fitting a wide range of nostril sizes for enhanced airtightness. Unlike existing invasive nasal masks, this patient interface device does not enter the user's nostrils, reducing sensory disturbances and making it more suitable for the majority of people. This feature makes it particularly suitable for most users, especially those new to using devices for sleep apnea treatment, as they find adapting to wearing such devices easier. Also, compared to existing patient interface devices that seal the area from the user's nose bridge to the upper lip region, this device has a smaller volume which means it's easier for users to wear and easier to adjust when the nasal mask shifts. This design lowers the risk of gas leakage to some extent, benefiting users by making the device more portable and convenient when traveling, thereby facilitating the continuity of treatment.
2) This disclosure takes an innovative method for noise reduction. The ventilation component in this disclosure uniquely incorporates noise reduction material onto the ventilation component mouth to disperse the airflow. Moreover, the configured distance between the noise reduction material and the port cover ensures a gentler flow of gas to the external environment. In existing patient interface devices available on the market, the way to use the noise reduction material involves placing a piece of noise reduction cotton between both ports of the ventilation component. Although the airflow is dispersed by the noise reduction cotton, due to the limited space between the cotton and both ports, the airflow becomes turbulent at both the air inlet and air outlet of the ventilation component, which is not conducive to the use by the user. Additionally, positioning the noise reduction cotton in a non-detachable manner between both ports of the ventilation component makes it challenging for users to clean. Innovatively, this disclosure positions the noise reduction material, including a noise reduction mesh set at the air inlet of the ventilation component, establishing a certain gap between the noise reduction material and the second port (which is also the port cover's location) of the ventilation component. As gas flows from the front chamber to the external environment through the ventilation component, it first disperses through the noise reduction material, then a turbulent airflow becomes a gentler flow due to the gap between the noise reduction material and the port cover, eventually exiting to the external environment through the gap between the port cover and the ventilation component. Compared to existing patient interface devices, such a process results in more apparent noise reduction and allows the airflow to be directed to the user in a non-frontal manner, reducing the possibility of airflow blowing onto the user's face. In summary, the ventilation component of the disclosure outperforms the current ventilation component of the patient interface in several aspects.
3) The design is modular, which expands the range of user choices. This disclosure introduces a modular design for the ventilation component. Compared to existing designs, the ventilation component of this disclosure allows for the replacement of the port cover and noise reduction material. Users can choose and replace the appropriate noise reduction material and user-friendly type of port cover to fit the ventilation component body in accordance with their preferences and needs. In this way, users are provided with diverse options. a. The port cover of the ventilation component in this disclosure is detachably connectable to the ventilation component. Furthermore, the noise reduction material is internally formed with the ventilation component body in a non-detachable manner, rather than being placed in the middle part of the component. As a result, the presence or absence of a port cover does not cause the noise reduction material to fall out due to a lack of obstruction, and it does not essentially impact the noise reduction effect. The primary role of the port cover in this design is to leave a gap between the ventilation component and the noise reduction material, turning the turbulent airflow that passes through the noise reduction material into a gentler flow to the external environment. However, even without the port cover, the airflow from the front chamber will still be dispersed through the noise reduction material, achieving the noise reduction effect. While having a port cover offers certain advantages, the port cover isn't the primary component providing noise reduction. Therefore, users can choose whether or not to use the port cover, and still achieve noise reduction. This provides an option for users to experience noise reduction without using the port cover at their own acceptance. b.

In scenarios where the port cover is used, the detachable port cover allows users to choose their preferred port cover and its connection methods, which include options like a fully detachable port cover or a flip port cover. Different port covers and connection methods have their pros and cons. Users can choose the fully detachable port cover for detailed cleaning of the interior of the ventilation component, or opt for the flip port cover, which is more convenient for users to snap-fit it in place and is less likely to fall off, avoiding situations where the port cover cannot be found. Both types of port covers are more conducive to cleaning and drying the noise reduction material compared to closed port covers. c. This disclosure has a replaceable design for the noise reduction material. Since the noise reduction material is integrated with the ventilation component body in a non-detachable manner, the mouth on one port of the ventilation component has a detachable port cover. The ventilation component, consisting of only two parts, facilitates a modular design. Therefore, this design allows the connection of the ventilation component body with different types or forms of noise reduction material to the same port cover. Users can choose ventilation components with different types or forms of noise reduction materials to meet their diverse needs for ventilation. This approach adds flexibility to the entire patient interface device product. The replaceable modules can cater to users' varying needs and preferences, reducing the complexity of the ventilation component.
4) This disclosure ensures a more thorough and hygienic cleaning. As the product comes in direct contact with the user's face and communicates with the user's airway, maintaining the hygiene of the respiratory pathway is needed for the user's health, which effectively reduces the risk of infection intake. a. The port cover on the outlet side of the patient interface device in the existing market is usually a non-detachable whole with the ventilation component, and the noise reduction material is placed inside the ventilation component on the inner side of the port cover, which is very unfavorable to the user's cleaning of the ventilation component. When users clean the internal noise reduction material (i.e., noise reduction cotton) or after prolonged use when moisture accumulates in the noise reduction cotton, the inseparability of the port cover means that only small gaps exist for drying the noise reduction cotton. This can easily lead to incomplete drying and then bacterial growth. However, this disclosure makes the port cover detachable, enabling a more thorough and cleaner cleaning by directly accessing the noise reduction material by opening the port cover. The port cover can also be opened for drying and ventilation, offering higher hygiene standards compared to existing products, which effectively reduces the risk of infection intake. Furthermore, the ventilation component of this disclosure has two layers: the noise reduction material on one port, the port cover on the other port. This design is overall more streamlined than the three-layer form available on the market with the noise reduction material in the middle part of the ventilation component, which facilitates the cleaning and disinfection of both the entire ventilation component and its details, leaving no narrow gaps uncleaned. b. In addition, the noise reduction material used in this disclosure can be a noise reduction mesh. The noise reduction cotton used in the current market is difficult to clean and dry due to the non-openable port cover, and it easily absorbs exhaled moisture, leading to the growth of bacteria and mold. In contrast, the noise reduction mesh material doesn't have these issues. It does not absorb moisture, and the port cover can be opened, allowing the noise reduction mesh to be scrubbed repeatedly with a brush, so that the ventilation component is more durable. The noise reduction mesh used in this disclosure can withstand repeated cleaning without getting damaged, offering more protection for the user's health. c. The double-layer wall technology available on the current market tends to accumulate dirt and is not easy to clean. Over time, users can be easily infected by bacteria and other microbes, posing a risk of illness. This disclosure, through the aforementioned foundational improvements, changes the sealing structure from the double-layer wall on the market to a single-layer wall structure. Not only does this present advantages in usage, but in terms of cleaning, compared to multi-layer wall structures, it is more straightforward. Users can clean the sealing structure more easily, without the internal crevices or hard-to-reach areas found in multi-layer wall structures, making cleaning quicker and more effective.
5) This disclosure has reduced processes, a lower cost, a decreased defect rate, and is environmentally friendly. The patient interface device in this disclosure differs from current products in both the sealing structure and the ventilation component. With the innovative improvements of this disclosure, apart from the aforementioned advantages, the product also has fewer production processes, lower manufacturing costs and a lower defect rate. Moreover, the improved technology results in a reduced material usage, benefiting the environment. a. The improvements mentioned above in this disclosure make the sealing structure change from a double wall in the market to a single-layer wall structure. From a manufacturing standpoint, the single-layer wall design is more cost-effective than the multi-layer wall, as the latter requires more material and a relatively more complex production process and mold cost. In comparison, the production process of the single-layer wall is more economical and efficient. This approach also conserves raw materials used in manufacturing the patient interface. By saving on material usage, there is a reduced demand for plastics, polyester, silicone, etc., leading to decreased carbon emissions. Continuing to adopt this green design approach will have a positive impact on a larger scale, taking a step nearer towards carbon neutrality. b. In this disclosure, the noise reduction material and the mouth of the ventilation component body where the airflow enters the ventilation component body from the front chamber are integrally formed, different from the current patient interface devices where components and noise reduction materials are produced separately and then assembled. And this design simplifies the production process by using the method of manufacturing a single, unified product without the need for assembly, which is faster compared to the manufacturing and assembling steps of existing products, thereby reducing production time, labor costs, production processes, and equipment requirements. The integral fabrication also enhances the structural strength of the ventilation component to a certain extent. Because parts are made and connected in one operation, the possibility of assembly errors is decreased. This method enhances product quality and reliability, making the product more durable, further realizing an environmentally friendly design.

The embodiments of the disclosure have been detailed in conjunction with the accompanying drawings. However, the scope of the disclosure is not limited to the specific embodiments outlined above. These embodiments are intended primarily for illustrative purposes, rather than as limitations. Those with ordinary skills in this field, inspired by the disclosure without departing from the scope of the disclosure's objectives and claims, can develop numerous variations. All such modifications are encompassed within the protective scope of this disclosure.

It must be noted that as used herein and in the appended claims, the singular forms “a”, “an”, and “the” include their plural equivalents, unless the context clearly dictates otherwise.

1. A patient interface device to deliver continuous positive pressure gas to a nasal airway of a user in a sealed manner for treatment of sleep disordered breathing, the patient interface device comprising:
a front chamber section, which includes a main chamber formed by a continuous wall and includes two entrance ends located on both sides, with the main chamber being curved towards a side nearer a face of the user to form a chamber structure with a certain curvature that does not make contact with the user;
a sealing structure, with a single-layer wall, which is configured to be two shaped protrusions and to communicate with the main chamber of the front chamber section, and at least a part of which, when in use, contacts a portion of a nose of the user and allows gas to be delivered through an exhaust end, to enter at least one nostril, thereby to complete a pressurized gas delivery and to seal the nasal airway of the user, with the two shaped protrusions being symmetrical to each other, wherein the exhaust end of each of the two shaped protrusions have a non-uniform wall thickness and the non-uniform wall thickness is greater than a wall thickness of an opening in the exhaust end such that when the patient interface device is worn, uniform pressure is provided around a periphery of the at least one nostril to seal the nasal airway of the user, and wherein the non-uniform wall thickness is configured to be uniformly variable;
wherein the front chamber section includes at least one opening, and a ventilation component is provided on one of the openings of the front chamber section, the ventilation component comprising:
a noise reduction material and a ventilation component body with two ports, wherein the noise reduction material forms an inseparable whole with one port of the ventilation component body, wherein a connection between the noise reduction material and the ventilation component body is seamless, wherein the noise reduction material first divides an airflow that exits from the front chamber section into smaller streams, before the airflow reaches an external environment through a mouth on the other port of the two ports of the ventilation component body away from the noise reduction material; and
a positioning stabilizer, configured to be provided on both ends of the front chamber section to provide a connection between components to secure the patient interface device to the face of the user.

2. The patient interface device according to claim 1, wherein the ventilation component includes a port cover connectable to the mouth on one port of the ventilation component body away from the noise reduction material, and a dispersed airflow from the ventilation component flows out to the external environment through a gap between a periphery of the port cover and the mouth of the ventilation component.

3. The patient interface device according to claim 1, wherein the noise reduction material and the ventilation component body are made of a same material.

4. The patient interface device according to claim 1, wherein the two protrusions are symmetrical themselves.

5. (canceled)

6. A patient interface device to deliver continuous positive pressure gas to a nasal airway of a user in a sealed manner for treatment of sleep disordered breathing, the patient interface device comprising:
a front chamber section, which includes a main chamber formed by a continuous wall and includes two entrance ends located on both sides, with the main chamber being curved towards a side nearer a face of the user to form a chamber structure with a certain curvature that does not make contact with the user;
a sealing structure, which is configured to be two shaped protrusions and to communicate with the main chamber of the front chamber section, and at least a part of which, when in use, contacts at least a portion of a nose of the user and allows gas to be delivered through an exhaust end to enter at least one nostril, thereby to complete a pressurized gas delivery and to seal the nasal airway of the user, wherein the exhaust end of each of the two shaped protrusions have a non-uniform wall thickness and the non-uniform wall thickness is greater than a wall thickness of an opening in the exhaust end, and wherein the non-uniform wall thickness is configured to be uniformly variable;
wherein the front chamber section includes at least one opening, and a ventilation component is provided on one of the openings of the front chamber section, the ventilation component comprising:
a noise reduction material, a ventilation component body with two ports, and a port cover, wherein the noise reduction material is integrally formed with one port of the ventilation component body to make an inseparable whole, wherein the port cover is positionable on the other port of the two ports of the ventilation component body away from the noise reduction material, wherein the noise reduction material, through which an airflow that exits from the front chamber section is dispersed via pores in the noise reduction material, is situated at a specific distance from the port cover such that the ventilation component body is configured such that the airflow, once dispersed by the noise reduction material, is able to gather more gently before being discharged into an external environment at a gas flow rate between 0.1 to 6 liters per second, to reduce noise production,
wherein there is a gap with a certain distance between a periphery of the port cover and a mouth of the ventilation component, configured to guide a direction of the airflow through the noise reduction material to facilitate disassembly, and wherein a positioning of the port cover results in different structures for an air inlet and an air outlet of the ventilation component; and
a positioning stabilizer, which is configured to be provided on both ends of the front chamber section to provide a connection between components to secure the patient interface device to the face of the user.

7. The patient interface device according to claim 6, wherein the noise reduction material and the ventilation component body are made of different materials.

8. The patient interface device according to claim 6, wherein the positioning stabilizer is harder than the front chamber section.

9. The patient interface device according to claim 6, wherein the port cover is connectable to the ventilation component body via a snap-fitting.

10. The patient interface device according to claim 6, wherein the ventilation component is detachably connectable to the opening of the front chamber section.

11. A patient interface device to deliver continuous positive pressure gas to a nasal airway of a user in a sealed manner for treatment of sleep disordered breathing, the patient interface device comprising:
a front chamber section, which includes a main chamber formed by a continuous wall and includes two entrance ends located on both sides, with the main chamber being curved towards a side nearer a face of the user to form a chamber structure with a certain curvature that does not make contact with the user;
a sealing structure, which is configured to be two shaped protrusions and to communicate with the main chamber of the front chamber section, and at least a part of which, when in use, contacts at least a portion of a nose of the user and allows gas to be delivered through an exhaust end to enter at least one nostril, thereby to complete a pressurized gas delivery and to seal the nasal airway of the user, wherein the exhaust end of each of the two shaped protrusions have a non-uniform wall thickness and the non-uniform wall thickness is greater than a wall thickness of an opening in the exhaust end, and wherein the non-uniform wall thickness is configured to be uniformly variable;
wherein the front chamber section includes at least one opening, and a ventilation component is provided on one of the openings of the front chamber section, and wherein the ventilation component comprises:
a noise reduction material and a ventilation component body with two ports, wherein the noise reduction material forms an inseparable whole with one port of the ventilation component body, wherein a connection between the noise reduction material and the ventilation component body is seamless, and wherein the noise reduction material is configured to first divide an airflow that exits from the front chamber section into smaller streams, before the airflow reaches an external environment through a mouth on the other port of the two ports of the ventilation component body away from the noise reduction material such that the ventilation component body is configured such that the airflow, once divided by the noise reduction material, is able to gather more gently before being discharged into the external environment at a gas flow rate between 0.1 to 6 liters per second to reduce noise production;
a positioning stabilizer, which is configured to be provided on both ends of the front chamber section to provide a connection between components to secure the patient interface device to the face of the user;
wherein an area of the opening of the front chamber section, at which the ventilation component is installed, has a ratio of at least 1:9 to an outer wall area of the front chamber section, and the area of the opening of the front chamber section has a ratio of at least 5:1 to an area of the noise reduction material; and
wherein the noise reduction material has at least one of the following features:
1) the noise reduction material being made of fibrous material;
2) a thickness of the noise reduction material being at or between 0.2 to 5 mm;
3) one side of the noise reduction material having a surface area of at least 50 mm².

12. The patient interface device according to claim 11, wherein the ventilation component includes a port cover connectable to the mouth on one port of the ventilation component body away from the noise reduction material, a dispersed airflow from the ventilation component flows out to the external environment through a gap between a periphery of the port cover and the mouth of the ventilation component.

13. The patient interface device according to claim 11, wherein a periphery of the ventilation component includes a clamping structure that connects to the front chamber section.

14. The patient interface device according to claim 11, wherein a shape of the noise reduction material is similar to a shape of the mouth connectable to the noise reduction material of the ventilation component body.

15. The patient interface device according to claim 12, wherein the mouth connectable to the noise reduction material of the ventilation component body is smaller than or equal to the mouth of the ventilation component body connectable to the port cover.

16. The patient interface device according to claim 11, wherein the positioning stabilizer extends in a direction of the openings at both ends of the front chamber section without bending itself.

17. A patient interface device to deliver continuous positive pressure gas to a nasal airway of a user in a sealed manner for treatment of sleep disordered breathing, the patient interface device comprising:
a front chamber section, which includes a main chamber formed by a continuous wall and includes two entrance ends located on both sides, with the main chamber being curved towards a side nearer a face of the user to form a chamber structure with a certain curvature that does not make contact with the user;
a sealing structure, with a single-layer wall, which is configured to be two shaped protrusions to communicate with the main chamber of the front chamber section, and at least a part of which, when in use, contacts a portion of a nose of the user and allows gas to be delivered through an exhaust end to enter at least one nostril, thereby to complete a pressurized gas delivery and to seal the nasal airway of the user, with the two shaped protrusions being symmetrical to each other, wherein the exhaust end of each of the two shaped protrusions have a non-uniform wall thickness and the non-uniform wall thickness is greater than a wall thickness of an opening in the exhaust end such that when the patient interface device is worn, uniform pressure is provided around a periphery of the at least one nostril to seal the nasal airway of the user, and wherein the non-uniform wall thickness is configured to be step-shaped;
the front chamber section having at least one opening, and a ventilation component provided on one of the openings of the front chamber section, the ventilation component comprising:
a noise reduction material and a ventilation component body with two ports, wherein the noise reduction material forms an inseparable whole with one port of the ventilation component body, wherein a connection between the noise reduction material and the ventilation component body is seamless, and wherein the noise reduction material first divides an airflow that exits from the front chamber section into smaller streams, before the airflow reaches an external environment through a mouth on the other port of the two ports of the ventilation component body away from the noise reduction material;
a positioning stabilizer, which is configured to be provided on both ends of the front chamber section to provide a connection between components to secure the patient interface device to the face of the user;
wherein an area of the opening of the front chamber section, at which the ventilation component is installed, has a ratio of at least 1:9 to an outer wall area of the front chamber section, and the area of the opening of the front chamber section has a ratio of at least 5:1 to an area of the noise reduction material; and
wherein the noise reduction material has at least one of the following features:
1) the noise reduction material being one of foam-type or granular-type material;
2) a thickness of the noise reduction material being at or between 0.5 to 7 mm;
3) one side of the noise reduction material having a surface area of at least 50 mm².

18. The patient interface device according to claim 17, wherein the ventilation component includes a port cover connectable to the mouth on one port of the ventilation component body away from the noise reduction material, a dispersed airflow from the ventilation component flows out to an external environment through a gap between a periphery of the port cover and the mouth of the ventilation component.

19. (canceled)

20. The patient interface device according to claim 17, wherein the protrusions of the sealing structure are formed by extending in a normal direction from a specific location of the front chamber section.

21. The patient interface device according to claim 17, wherein each center of two exhaust ends on the sealing structure that contact the face of the user, is equidistant from a center of the noise reduction material.

22. The patient interface device according to claim 17, wherein the sealing structure has a tendency to converge towards a point of its center.
